(19) United States
(12) Patent Application Publication
(10) Pub. No.: US 2014/0056823 A1
(43) Pub. Date: Feb. 27, 2014

(54) COMBINATIONS OF MEDICAMENTS FOR THE TREATMENT OF RESPIRATORY DISEASES

(71) Applicants: Thierry BOUYSSOU, Warthausen (DE); Michael P. PIEPER, Biberach an der Riss (DE); Andreas SCHNAPP, Biberach an der Riss (DE)

(72) Inventors: Thierry BOUYSSOU, Warthausen (DE); Michael P. PIEPER, Biberach an der Riss (DE); Andreas SCHNAPP, Biberach an der Riss (DE)

(73) Assignee: BOEHRINGER INGELHEIM PHARMA GMBH & CO. KG, Ingelheim am Rhein (DE)

(21) Appl. No.: 14/071,035

(22) Filed: Nov. 4, 2013

Related U.S. Application Data
(63) Continuation of application No. 13/796,445, filed on Mar. 12, 2013, now abandoned.
Application No. 13/796,445 is a continuation of application No. 13/537,589, filed on Jun. 29, 2012, now abandoned.
Application No. 13/537,589 is a continuation of application No. 13/028,434, filed on Feb. 16, 2011, now abandoned.
Application No. 13/028,434 is a continuation of application No. 11/543,694, filed on Oct. 5, 2006, now abandoned.

(30) Foreign Application Priority Data
Oct. 10, 2005 (EP) 05109374.8

(51) Int. Cl. A61K 31/538; A61K 31/439

(52) U.S. Cl. CPC A61K 31/538; A61K 31/439
USPC 424/45; 514/230.5

(57) ABSTRACT

A pharmaceutical composition comprising:
(a) a compound of formula 1

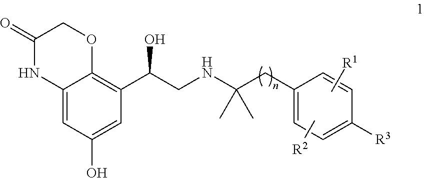

wherein:
n is 1 or 2;
R¹ is hydrogen, halogen, C₁₋₄-alkyl, or O—C₁₋₄-alkyl;
R² is hydrogen, halogen, C₁₋₄-alkyl, or O—C₁₋₄-alkyl; and
R³ is hydrogen, C₁₋₄-alkyl, OH, halogen, O—C₁₋₄-alkyl, O—C₁₋₄-alkylene-COOH, or O—C₁₋₄-alkylene-COO—C₁₋₄-alkyl,
   or an enantiomer, mixture of enantiomers, or racemate thereof, or an acid addition salt with pharmacologically acceptable acids thereof, or a solvate or hydrate thereof; and
(b) another active substance 2,
   wherein the molar ratio of the compound of formula 1 to the active substance 2 is 1:10 to 12:1.

### RELATED APPLICATIONS

This application claims priority to European Application No. 05109374.8, filed Oct. 10, 2005, which is hereby incorporated by reference.

### FIELD OF THE INVENTION

The present invention relates to new medicament combinations, which contain, in addition to one or more, preferably one compound of general formula 1

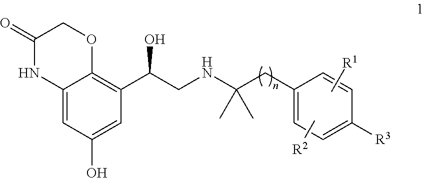

wherein the groups R¹, R², and R³ may have the meanings given in the claims and in the specification, at least one other active substance 2, processes for preparing them, and their use as pharmaceutical compositions.

### BACKGROUND OF THE INVENTION

The present invention relates to novel pharmaceutical compositions based on long-acting anticholinergic compounds and long-acting β-mimetics, processes for preparing them, and their use in treating respiratory complaints.

It is known from the prior art that β-mimetics and anticholinergics may be used successfully in combination as bronchospasmolytics for the treatment of obstructive respiratory complaints, such as, e.g., asthma or chronic obstructive pulmonary disease. For drug treatment of diseases it is often desirable to prepare medicaments with a longer duration of activity. As a rule, this ensures that the concentration of the active substance in the body needed to achieve the therapeutic effect is present over a longer period of time without the need to administer the drug repeatedly, frequently. The administration of an active substance at longer intervals of time also contributes considerably to the patient's wellbeing. It is particularly desirable to provide a pharmaceutical composition which can be used to therapeutically good effect by administering it once a day (single dose). A single application per day has the advantage that the patient can become accustomed relatively quickly to the regular taking of the medicament at a particular time of the day.

However, the administration of substances with a β-sympathomimetic activity, such as e.g., the active substance formoterol which is also known from the prior art, may be associated with undesirable side effects in humans.

Examples of central effects include general malaise, agitation, insomnia, anxiety, trembling fingers, sweating and headaches. Administration by inhalation does not eliminate these side effects, but generally they are somewhat less severe than after oral or parenteral administration.

The side effects of the β-sympathomimetics after administration by inhalation are, however, based predominantly on the more or less marked β1-stimulant effects on the heart. After systemic availability, β-sympathomimetics give rise to tachycardia, palpitations, angina pectoris-like pain as well as arrhythmias [Jackson and Lipworth, Drug Safety 2004: 24, 243-270; Sovani et al., Drug Safety 2004: 27, 689-715].

The aim of the present invention is therefore to provide novel pharmaceutical compositions based on anticholinergic compounds and long-acting β-mimetics, which on the one hand have a therapeutic benefit in the treatment of respiratory complaints and are characterized by a long duration of activity, while simultaneously reducing the potential for side effects of the β-mimetic and may thus be used to prepare pharmaceutical compositions with a longer-lasting activity and a low side effect profile.

Surprisingly it has been found that the aims outlined above can be achieved by means of compounds of general formula 1.

### DETAILED DESCRIPTION OF THE INVENTION

The present invention relates to new medicament combinations which contain, in addition to one or more, preferably one compound of general formula 1

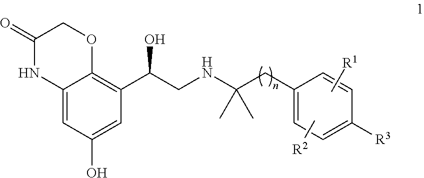

wherein:
n denotes 1 or 2;
R¹ denotes hydrogen, halogen, C₁₋₄-alkyl, or O—C₁₋₄-alkyl;
R² denotes hydrogen, halogen, C₁₋₄-alkyl, or O—C₁₋₄-alkyl; and
R³ denotes hydrogen, C₁₋₄-alkyl, OH, halogen, O—C₁₋₄-alkyl, O—C₁₋₄-alkylene-COOH, or O—C₁₋₄-alkylene-COO—C₁₋₄-alkyl,
   at least one other active substance 2, the molar ratio of the active substances 1 to 2 being in the range from 1:10 to 12:1, preferably 1:10 to 10:1.

Preferred are medicament combinations which contain, in addition to one or more, is preferably one compound of general formula 1, wherein:
n denotes 1 or 2;
R¹ denotes hydrogen, halogen or C₁₋₄-alkyl;
R² denotes hydrogen, halogen or C₁₋₄-alkyl; and
R³ denotes hydrogen, C₁₋₄-alkyl, OH, halogen, O—C₁₋₄-alkyl, O—C₁₋₄-alkylene-COOH or O—C₁₋₄-alkylene-COO—C₁₋₄-alkyl,
   at least one other active substance 2, the molar ratio of the active substances 1 to 2 being in the range from 1:10 to 12:1, preferably 1:10 to 10:1.

Also preferred are medicament combinations which contain, in addition to one or more, preferably one compound of general formula 1, wherein:
n denotes 1 or 2;
R¹ denotes hydrogen, fluorine, chlorine, or methyl;
R² denotes hydrogen, fluorine, chlorine, or methyl;
R³ denotes hydrogen, C₁₋₄-alkyl, OH, fluorine, chlorine, bromine, O—C₁₋₄-alkyl, O—C₁₋₄-alkylene-C 00H, or O—C₁₋₄-alkylene-COO—C₁₋₄-alkyl;
   at least one other active substance 2, the molar ratio of the active substances 1 to 2 being in the range from 1:10 to 12:1, preferably 1:10 to 10:1.

Also preferred are medicament combinations which contain, in addition to one or more, preferably one compound of general formula 1, wherein:
n denotes 1 or 2;
R¹ denotes hydrogen, methyl or ethyl;
R² denotes hydrogen, methyl or ethyl; and
R³ denotes hydrogen, methyl, ethyl, OH, methoxy, ethoxy, O—CH₂—COOH, O—CH₂—COO-methyl, or O—CH₂—COO-ethyl,
   at least one other active substance 2, the molar ratio of the active substances 1 to 2 being in is the range from 1:10 to 12:1, preferably 1:10 to 10:1.

Also preferred are medicament combinations which contain, in addition to one or more, preferably one compound of general formula 1, wherein:

n denotes 1 or 2;
R¹ denotes hydrogen or methyl;
R² denotes hydrogen or methyl; and
R³ denotes hydrogen, methyl, OH, methoxy, O—CH₂—COOH, or O—CH₂—COO-ethyl,
at least one other active substance 2, the molar ratio of the active substances 1 to 2 being in the range from 1:10 to 12:1, preferably 1:10 to 10:1.

In another preferred aspect the present invention relates to medicament combinations which contain, in addition to one or more, preferably one compound of general formula 1, wherein n=1, R¹ and R² denote hydrogen and the group R³ may have the meanings given above, at least one other active substance 2, the molar ratio of the active substances 1 to 2 being in the range from 1:10 to 12:1, preferably 1:10 to 10:1.

In the compounds of formula 1 the groups R¹ and R², if they do not represent hydrogen, may in each case be in the ortho or meta position relative to the link to the benzylic —CH₂-group. If none of the groups R¹ and R² denotes hydrogen, the preferred compounds of formula 1 for the medicament combinations according to the invention are those wherein the two groups R¹ and R² are either in the ortho configuration or both groups R¹ and R² are in the meta configuration, while compounds wherein both groups R¹ and R² are in the ortho configuration are of particular importance. In the compounds of formula 1 wherein one of the groups R¹ and R² does not denote hydrogen, this may be in the ortho or meta configuration relative to the link to the benzylic —CH₂— group. In this case, particularly preferred compounds of formula 1 for the medicament combinations according to the invention are those wherein the group R¹ or R², which does not denote hydrogen, is in the ortho configuration.

Particularly preferred are medicament combinations which contain, in addition to one or more, preferably one compound of general formula 1 selected from the compounds
6-hydroxy-8-{1-hydroxy-2-[2-(4-methoxyphenyl)-1,1-dimethylethylamino]ethyl}-4H-benzo[1,4]oxazin-3-one (1.1);
8-{2-[2-(2,4-difluorophenyl)-1,1-dimethylethylamino]-1-hydroxyethyl}-6-hydroxy-4H-benzo[1,4]oxazin-3-one (1.2);
8-{2-[2-(3,5-difluorophenyl)-1,1-dimethylethylamino]-1-hydroxyethyl}-6-hydroxy-4H-benzo[1,4]oxazin-3-one (1.3);
8-{2-[2-(4-ethoxyphenyl)-1,1-dimethylethylamino]-1-hydroxyethyl}-6-hydroxy-4H-benzo[1,4]oxazin-3-one (1.4);
8-{2-[2-(4-fluorophenyl)-1,1-dimethylethylamino]-1-hydroxyethyl}-6-hydroxy-4H-benzo[1,4]oxazin-3-one (1.5),
   at least one other active substance 2, the molar ratio of the active substances 1 to 2 being in the range from 1:10 to 12:1, preferably 1:10 to 10:1.

In the medicament combinations according to the invention, the compounds of formula 1 may be present in the form of the individual optical isomers, mixtures of the individual enantiomers or racemates. Particularly preferred are those medicament combinations in which one or more, preferably one compound of formula 1 is present in the form of the enantiomerically pure compounds, preferably in the form of the R-enantiomers. Methods of separating racemates into the respective enantiomers are known in the prior art and may be used analogously to prepare the enantiomerically pure R- and S-enantiomers of the compounds of formula 1. In another aspect, the present invention relates to medicament combinations which contain the abovementioned compounds of formula 1 in the form of the acid addition salts with pharmacologically acceptable acids as well as optionally in the form of the solvates and/or hydrates thereof.

In the medicament combinations according to the invention, the active substance 2 is selected from among the anticholinergics consisting of tiotropium salts (2.1), oxitropium is salts (2.2), flutropium salts (2.3), ipratropium salts (2.4), glycopyrronium salts (2.5), and trospium salts (2.6).

The abovementioned anticholinergics may optionally have chiral carbon centers. In this case, the medicament combinations according to the invention may contain the anticholinergics in the form of their enantiomers, mixtures of enantiomers or racemates, while preferably enantiomerically pure anticholinergics are used.

In the abovementioned salts 2.1 to 2.6 the cations tiotropium, oxitropium, flutropium, ipratropium, glycopyrronium, and trospium are the pharmacologically active constituents. An explicit reference to the abovementioned cations is indicated by the designations 2.1° to 2.6°. Any reference to the abovementioned salts 2.1 to 2.6 naturally also includes a reference to the corresponding cations tiotropium (2.1°), oxitropium (2.2°), flutropium (2.3°), ipratropium (2.4°), glycopyrronium (2.5°), trospium (2.6°).

By the salts 2.1 to 2.6 are meant according to the invention those compounds which contain, in addition to the cations tiotropium (2.1°), oxitropium (2.2°), flutropium (2.3°), ipratropium (2.4°), glycopyrronium (2.5°), and trospium (2.6°) chloride, bromide, iodide, sulfate, phosphate, methanesulfonate, nitrate, maleate, acetate, citrate, fumarate, tartrate, oxalate, succinate, benzoate, or p-toluenesulfonate as counter-ion (anion), the preferred counter-ions being chloride, bromide, iodide, sulfate, methanesulfonate, or p-toluenesulfonate. Of all the salts, the chlorides, bromides, iodide, and methanesulfonates are particularly preferred.

In the case of the trospium salts (2.6), the chloride is particularly preferred. In the case of the other salts 2.2 to 2.6, the methanesulfonates and bromides are of particular significance. Of particular importance are pharmaceutical combinations which contain tiotropium salts (2.1), oxitropium salts (2.2), or ipratropium salts (2.4), the associated bromides being of particular importance according to the invention. Tiotropium bromide is (2.1) is of particular importance.

The abovementioned salts may optionally be present in the drug combinations according to the invention in the form of their solvates or hydrates, preferably in the form of their hydrates. In the case of tiotropium bromide, the drug combinations according to the invention preferably contain it in the form of the crystalline tiotropium bromide monohydrate, which is known from WO 02/30928. If tiotropium bromide is used in anhydrous form in the drug combinations according to the invention, anhydrous crystalline tiotropium bromide is preferably used, which is known from WO 03/000265.

Examples of preferred medicament combinations of preferred compounds of formula 1 according to the invention with the abovementioned anticholinergics 2.1 to 2.6 are combinations containing the compounds 1.1 and 2.1; 1.1 and 2.2; 1.1 and 2.3; 1.1 and 2.4; 1.1 and 2.5; 1.1 and 2.6; 1.2 and 2.1; 1.2 and 2.2; 1.2 and 2.3; 1.2 and 2.4; 1.2 and 2.5; 1.2 and 2.6; 1.3 and 2.1; 1.3 and 2.2; 1.3 and 2.3; 1.3 and 2.4; 1.3 and 2.5; 1.3 and 2.6; 1.4 and 2.1; 1.4 and 2.2; 1.4 and 2.3; 1.4 and 2.4; 1.4 and 2.5; 1.4 and 2.6; 1.5 and 2.1; 1.5 and 2.2; 1.5 and 2.3; 1.5 and 2.4; 1.5 and 2.5; 1.5 and 2.6; in each case optionally in the form of their racemates, enantiomers, or diastereomers, and optionally in the form of their pharmacologically acceptable acid addition salts, solvates, and/or hydrates.

According to the invention, the molar ratio of the active substance 1 to the active substance 2 is preferably 1:1 to 12:1, particularly preferably 3:1 to 12:1, most particularly 5:1 to 12:1. According to the invention the molar ratio of the active substance 1 to the active substance 2 is preferably 1:1 to 10:1, particularly preferably 3:1 to 10:1, particularly 5:1 to 10:1. Preferred ranges for medicament combinations of the compounds of formula 1 according to the invention with the abovementioned anticholinergics 2.1 to 2.6 have the molar ratios listed in Table 1.

TABLE 1

Molar ratio of the
active substances 1:2

| Example # | from | to |
|---|---|---|
| 1 | 1.0:1 | 1.5:1 |
| 2 | 1.6:1 | 2.0:1 |
| 3 | 2.1:1 | 2.5:1 |
| 4 | 2.6:1 | 3.0:1 |
| 5 | 3.1:1 | 3.5:1 |
| 6 | 3.6:1 | 4.0:1 |
| 7 | 4.1:1 | 4.5:1 |
| 8 | 4.6:1 | 5.0:1 |
| 9 | 5.1:1 | 5.5:1 |
| 10 | 5.6:1 | 6.0:1 |
| 11 | 6.1:1 | 6.5:1 |
| 12 | 6.6:1 | 7.0:1 |
| 13 | 7.1:1 | 7.5:1 |
| 14 | 7.6:1 | 8.0:1 |
| 15 | 8.1:1 | 8.5:1 |
| 16 | 8.6:1 | 9.0:1 |
| 17 | 9.1:1 | 9.5:1 |
| 18 | 9.6:1 | 10.0:1 |
| 19 | 10.1:1 | 10.5:1 |
| 20 | 10.6:1 | 11.0:1 |
| 21 | 11.1:1 | 11.5:1 |
| 22 | 11.6:1 | 12.0:1 |

In a particularly preferred variant of the invention, inhalable pharmaceutical formulations of the medicament combinations according to the invention based on 10 ng of the bromide of 2.1 in the form of its monohydrate may contain the following amounts of the active substance 1 in the form of the hydrochloride thereof: 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and 10.0 ng.

In addition, inhalable pharmaceutical formulations of the medicament combinations according to the invention based on 5 ng of the bromide of 2.1 in the form of its monohydrate may contain the following amounts of the active substance 1 in the form of is the hydrochloride thereof: 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and 10.0 ng; particularly preferably 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0 ng of the active substance 1 in the form of the hydrochloride thereof.

### TERMS AND DEFINITIONS USED

By the term “C₁₋₄-alkyl” (including those which are part of other groups) are meant branched and unbranched alkyl groups with 1 to 4 carbon atoms. Examples of these include: methyl, ethyl, n-propyl, isopropyl, n-butyl, isobutyl, sec-butyl or tert-butyl. The abbreviations Me, Et, n-Pr, i-Pr, n-Bu, i-Bu, t-Bu, etc. may optionally also be used for the abovementioned groups. Unless stated otherwise, the definitions propyl and butyl include all the possible isomeric forms of the groups in question. Thus, for example, propyl includes n-propyl and isopropyl, butyl includes isobutyl, sec-butyl, and tert-butyl etc.

By the term “C₁₋₄-alkylene” (including those which are part of other groups) are meant branched and unbranched alkylene groups with 1 to 4 carbon atoms. Examples of these include: methylene, ethylene, propylene, 1-methylethylene, butylene, 1-methylpropylene, 1,1-dimethylethylene, or 1,2-dimethylethylene. Unless stated otherwise, the definitions is propylene and butylene include all the possible isomeric forms of the groups in question with the same number of carbons. Thus, for example, propyl also includes 1-methylethylene and butylene includes 1-methylpropylene, 1,1-dimethylethylene, and 1,2-dimethylethylene.

Halogen within the scope of the present invention denotes fluorine, chlorine, bromine, or iodine. Unless stated otherwise, fluorine, chlorine, and bromine are the preferred halogens.

By acid addition salts with pharmacologically acceptable acids are meant, for example, salts selected from the group comprising the hydrochloride, hydrobromide, hydroiodide, hydrosulfate, hydrophosphate, hydromethanesulfonate, hydronitrate, hydromaleate, hydroacetate, hydrobenzoate, hydrocitrate, hydrofumarate, hydrotartrate, hydrooxalate, hydrosuccinate, hydrobenzoate, and hydro-p-toluenesulfonate, preferably the hydrochloride, hydrobromide, hydrosulfate, hydrophosphate, hydrofumarate, and hydromethanesulfonate. Of the abovementioned acid addition salts, the salts of hydrochloric acid, methanesulfonic acid, benzoic acid, and acetic acid are particularly preferred according to the invention.

Within the scope of the present invention the expression medicament combination of components 1 and 2 denotes the joint administration of both active substances in a single preparation or formulation or the separate administration of the two active substances in separate formulations. If the active substances 1 and 2 are administered in separate formulations, this separate administration may be carried out simultaneously or at staggered times, i.e., sequentially.

### Ranges of Indications

In one aspect, the present invention relates to the abovementioned medicament combinations which contain in addition to therapeutically effective amounts of 1 and 2 a pharmaceutically acceptable carrier. In one aspect, the present invention relates to the is abovementioned pharmaceutical compositions which do not contain a pharmaceutically acceptable carrier in addition to therapeutically effective amounts of 1 and 2.

The present invention also relates to the use of therapeutically effective amounts of the active substances 1 for preparing a pharmaceutical composition also containing one or more, preferably one active substance 2 for the treatment of inflammatory and obstructive respiratory complaints, for inhibiting premature labor in midwifery (tocolysis), for restoring sinus rhythm in the heart in atrioventricular block, for correcting bradycardic heart rhythm disorders (antiarrhythmic), for treating circulatory shock (vasodilatation and increasing the heart volume), as well as for the treatment of skin irritations and inflammation.

In a preferred aspect, the present invention relates to the use of therapeutically effective amounts of the active substance 1 for preparing a pharmaceutical composition also containing one or more, preferably one, active substance 2 for the treatment of respiratory complaints selected from the group comprising obstructive pulmonary diseases of various origins, pulmonary emphysema of various origins, restrictive pulmonary diseases, interstitial pulmonary diseases, cystic fibrosis, bronchitis of various origins, bronchiectasis, ARDS (adult respiratory distress syndrome), and all forms of pulmonary edema.

Preferably the medicament combinations according to the invention are used as specified above for preparing a pharmaceutical composition for the treatment of obstructive pulmonary diseases selected from among bronchial asthma, pediatric asthma, severe asthma, acute asthma attacks, chronic bronchitis, and COPD (chronic obstructive pulmonary disease), while it is particularly preferable according to the invention to use them for preparing a pharmaceutical composition for the treatment of bronchial asthma and COPD.

It is also preferable to use the medicament combinations according to the invention for preparing a pharmaceutical composition for the treatment of pulmonary emphysema which is has its origins in COPD or al-proteinase inhibitor deficiency.

It is also preferable to use the medicament combinations according to the invention for preparing a pharmaceutical composition for the treatment of restrictive pulmonary diseases selected from among allergic alveolitis, restrictive pulmonary diseases triggered by work-related noxious substances, such as asbestosis or silicosis, and restriction caused by lung tumors, such as, for example, lymphangiosis carcinomatosa, bronchoalveolar carcinoma, and lymphomas.

It is also preferable to use the medicament combinations according to the invention for preparing a pharmaceutical composition for the treatment of interstitial pulmonary diseases selected from among pneumonia caused by infections, such as, for example, infection by viruses, bacteria, fungi, protozoa, helminths, or other pathogens, pneumonitis caused by various factors, such as, for example, aspiration and left heart insufficiency, radiation-induced pneumonitis, or fibrosis, collagenoses, such as, for example, lupus erythematodes, systemic sclerodermy, or sarcoidosis, granulomatoses, such as, for example, Boeck's disease, idiopathic interstitial pneumonia, or idiopathic pulmonary fibrosis (IPF).

It is also preferable to use the medicament combinations according to the invention for preparing a pharmaceutical composition for the treatment of cystic fibrosis or mucoviscidosis.

It is also preferable to use the medicament combinations according to the invention for preparing a pharmaceutical composition for the treatment of bronchitis, such as, for example, bronchitis caused by bacterial or viral infection, allergic bronchitis, and toxic bronchitis.

It is also preferable to use the medicament combinations according to the invention for preparing a pharmaceutical composition for the treatment of bronchiectasis.

It is also preferable to use the medicament combinations according to the invention for preparing a pharmaceutical composition for the treatment of ARDS (adult respiratory distress syndrome).

It is also preferable to use the medicament combinations according to the invention for preparing a pharmaceutical composition for the treatment of pulmonary edema, for example, toxic pulmonary edema after aspiration or inhalation of toxic substances and foreign substances.

It is particularly preferable to use the compounds detailed above for preparing a pharmaceutical composition for the treatment of asthma or COPD. Also of particular importance is the abovementioned use of medicament combinations according to the invention for preparing a pharmaceutical composition for once-a-day treatment of inflammatory and obstructive respiratory complaints, particularly for the once-a-day treatment of asthma or COPD.

The present invention also relates to the use of therapeutically effective amounts of an active substance of formula 1 in combination with therapeutically effective amounts of active substance 2 for preparing a pharmaceutical composition for the treatment of one of the abovementioned diseases.

The present invention also relates to a process for treating one of the abovementioned diseases, which is characterized in that therapeutically effective amounts of an active substance of formula 1 are administered in combination with therapeutically effective amounts of an active substance 2.

### Formulations

The two active substance components 1 and 2 may be administered, together or separately, in each case by inhalation or by oral, parenteral or some other route, in known manner, in substantially conventional formulations such as for example plain or coated tablets, pills, is granules, aerosols, syrups, emulsions, suspensions, powders, and solutions, using inert, non-toxic, pharmaceutically suitable carriers or solvents.

Suitable preparations for administering the compounds of formula 1 and 2 include tablets, capsules, suppositories, solutions, powders, etc. The proportion of pharmaceutically active compound or compounds should be in the range from 0.05% to 90% by weight, preferably 0.1% to 50% by weight of the total composition. Suitable tablets may be obtained, for example, by mixing the active substance(s) with known excipients, for example, inert diluents such as calcium carbonate, calcium phosphate, or lactose, disintegrants such as corn starch or alginic acid, binders such as starch or gelatin, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example, collidone or shellac, gum arabic, talc, titanium dioxide, or sugar. To achieve delayed release or prevent incompatibilities, the core may also consist of a number of layers. Similarly the tablet coating may consist of a number or layers to achieve delayed release, possibly using the excipients mentioned above for the tablets.

Syrups or elixirs containing the active substances or combinations of active substances according to the invention may additionally contain a sweetener such as saccharine, cyclamate, glycerol, or sugar and a flavor enhancer, e.g., a flavoring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions are prepared in the usual way, e.g., with the addition of isotonic agents, preservatives such as p-hydroxybenzoates, or stabilizers such as alkali metal salts of ethylenediamine tetraacetic acid, optionally using emulsifiers and/or dispersants, wherein if water is used as the diluent, for example, organic solvents may optionally be used as solvating agents or dissolving aids, and transferred into injection vials or ampoules or infusion bottles.

Capsules containing one or more active substances or combinations of active substances may, for example, be prepared by mixing the active substances with inert carriers such as lactose or sorbitol and packing them into gelatin capsules.

Suitable suppositories may be made for example by mixing with carriers provided for this purpose, such as neutral fats or polyethyleneglycol or the derivatives thereof.

Excipients which may be used include, for example, water, pharmaceutically acceptable organic solvents such as paraffins (e.g., petroleum fractions), vegetable oils (e.g., groundnut or sesame oil), mono- or polyfunctional alcohols (e.g., ethanol or glycerol), carriers such as, e.g., natural mineral powders (e.g., kaolins, clays, talc, chalk), synthetic mineral powders (e.g., highly dispersed silicic acid and silicates), sugars (e.g., cane sugar, lactose and glucose), emulsifiers (e.g., lignin, spent sulfite liquors, methylcellulose, starch and polyvinylpyrrolidone) and lubricants (e.g., magnesium stearate, talc, stearic acid and sodium lauryl sulfate).

For oral administration the tablets may, of course, contain, apart from the abovementioned carriers, additives such as sodium citrate, calcium carbonate, and dicalcium phosphate together with various additives such as starch, preferably potato starch, gelatin, and the like. Moreover, lubricants such as magnesium stearate, sodium lauryl sulfate, and talc may be used at the same time for the tabletting process. In the case of aqueous suspensions, the active substances may be combined with various flavor enhancers or colorings in addition to the excipients mentioned above.

Preferably, even when the two components 1 and 2 are administered separately, at least is component 1 is administered by inhalation. If component 1 is administered by inhalation, when the two active substances are taken separately, component 2 may also be administered, for example, by oral or parenteral route using formulations conventional in the art such as plain or coated tablets, pills, granules, aerosols, syrups, emulsions, suspensions, powders and solutions, using inert, non-toxic, pharmaceutically suitable carriers or solvents.

Preferably, however, the medicament combinations according to the invention are administered by inhalation by means of a single preparation containing both active substances 1 and 2 or by means of separate preparations each containing only one of the active substances 1 and 2, suitable for administration by inhalation.

Inhalable preparations include inhalable powders, propellant-containing metered dose aerosols or propellant-free inhalable solutions. Inhalable powders according to the invention containing the combination of active substances 1 and 2 may consist of the active substances on their own or of a mixture of the active substances with physiologically acceptable excipients. Within the scope of the present invention, the term propellant-free inhalable solutions also includes concentrates or sterile inhalable solutions ready for use. The preparations according to the invention may contain the combination of active substances 1 and 2 either together in one formulation or in two separate formulations. These formulations which may be used within the scope of the present invention are described in more detail in the next part of the specification.

### A) Inhalable Powder Containing the Combinations of Active Substances According to the Invention

The inhalable powders according to the invention may contain 1 and 2 either on their own or in admixture with suitable physiologically acceptable excipients. If the active substances 1 and 2 are present in admixture with physiologically acceptable excipients, the following physiologically acceptable excipients may be used to prepare these inhalable powders according to the invention: monosaccharides (e.g., glucose or arabinose), is disaccharides (e.g., lactose, saccharose, maltose, trehalose), oligo- and polysaccharides (e.g., dextrans), polyalcohols (e.g., sorbitol, mannitol, xylitol), salts (e.g., sodium chloride, calcium carbonate) or mixtures of these excipients with one another. Preferably, mono- or disaccharides are used, while the use of lactose, trehalose or glucose is preferred, particularly, but not exclusively, in the form of their hydrates.

Within the scope of the inhalable powders according to the invention the excipients have a maximum average particle size of up to 250 μm, preferably between 10 and 150 μm, most preferably between 15 and 80 μm. It may sometimes seem appropriate to add finer excipient fractions with an average particle size of 1 to 9 μm to the excipients mentioned above. These finer excipients are also selected from the group of possible excipients listed hereinbefore. Finally, in order to prepare the inhalable powders according to the invention, micronized active substance 1 and 2, preferably with an average particle size of 0.5 to 10 μm, more preferably from 1 to 6 μm, is added to the excipient mixture. Processes for producing the inhalable powders according to the invention by grinding and micronizing and finally mixing the ingredients together are known from the prior art. The inhalable powders according to the invention may be prepared and administered either in the form of a single powder mixture which contains both 1 and 2 or in the form of separate inhalable powders which contain only 1 or 2.

The inhalable powders according to the invention may be administered using inhalers known from the prior art. Inhalable powders according to the invention which contain a physiologically acceptable excipient in addition to 1 and 2 may be administered, for example, by means of inhalers which deliver a single dose from a supply using a measuring chamber as described in U.S. Pat. No. 4,570,630, or by other means as described in DE 36 25 685 A. The inhalable powders according to the invention which contain 1 and 2 optionally in conjunction with a physiologically acceptable excipient may be administered, for example, using the inhaler known by the name TURBOHALER® or using inhalers as disclosed for example in EP 237507A. Preferably, the inhalable powders according to the invention which contain physiologically acceptable excipient in addition is to 1 and 2 are packed into capsules (to produce so-called inhalettes) which are used in inhalers as described, for example, in WO 94/28958.

A particularly preferred inhaler for using the pharmaceutical combination according to the invention in inhalettes is shown in FIG. 1. This inhaler (HANDIHALER®) for inhaling powdered pharmaceutical compositions from capsules is characterized by a housing 1 containing two windows 2, a deck 3 in which there are air inlet ports and which is provided with a screen 5 secured by a screen housing 4, an inhalation chamber 6 connected to the deck 3 on which there is a push button 9 provided with two sharpened pins 7 and movable counter to a spring 8, and a mouthpiece 12 which is connected to the housing 1, the deck 3 and a cover 11 via a spindle 10 to enable it to be flipped open or shut, and air through-holes 13 for adjusting the flow resistance.

If the inhalable powders according to the invention are to be packaged in capsules, in accordance with the preferred method of administration described above, the capsules should preferably contain from 1 to 30 mg each. According to the invention they contain either together or separately the dosages per single dose specified for 1 and 2 hereinbefore.

### B) Propellant Gas-Driven Inhalation Aerosols Containing the Combinations of Active Substances According to the Invention

Inhalation aerosols containing propellant gas according to the invention may contain substances 1 and 2 dissolved in the propellant gas or in dispersed form. 1 and 2 may be present in separate formulations or in a single preparation, in which 1 and 2 are either both dissolved, both dispersed or only one component is dissolved and the other is dispersed. The propellant gases which may be used to prepare the inhalation aerosols according to the invention are known from the prior art. Suitable propellant gases are selected from among hydrocarbons such as n-propane, n-butane or isobutane and halohydrocarbons such as preferably chlorinated and fluorinated derivatives of methane, ethane, propane, butane, cyclopropane or cyclobutane. The propellant gases mentioned above may be used on their is own or in mixtures thereof. Particularly preferred propellant gases are halogenated alkane derivatives selected from TG11, TG12, TG134a (1,1,1,2-tetrafluoroethane), TG227 (1,1,1,2,3,3,3-heptafluoropropane) and mixtures thereof, the propellant gases TG134a, TG227 and mixtures thereof being preferred.

The propellant-driven inhalation aerosols according to the invention may also contain other ingredients such as co-solvents, stabilizers, surfactants, antioxidants, lubricants and pH adjusters. All these ingredients are known in the art.

The inhalation aerosols containing propellant gas according to the invention may contain up to 5 wt.-% of active substance 1 and/or 2. Aerosols according to the invention contain, for example, 0.002 to 5 wt.-%, 0.01 to 3 wt.-%, 0.015 to 2 wt.-%, 0.1 to 2 wt.-%, 0.5 to 2 wt.-% or 0.5 to 1 wt.-% of active substance 1 and/or 2.

If the active substances 1 and/or 2 are present in dispersed form, the particles of active substance preferably have an average particle size of up to 10 μm, preferably from 0.1 to 6 μm, more preferably from 1 to 5 μm.

The propellant-driven inhalation aerosols according to the invention mentioned above may be administered using inhalers known in the art (MDIs=metered dose inhalers). Accordingly, in another aspect, the present invention relates to pharmaceutical compositions in the form of propellant-driven aerosols as hereinbefore described combined with one or more inhalers suitable for administering these aerosols. In addition, the present invention relates to inhalers which are characterized in that they contain the propellant gas-containing aerosols described above according to the invention. The present invention also relates to cartridges which are fitted with a suitable valve and can be used in a suitable inhaler and which contain one of the abovementioned propellant gas-containing inhalation aerosols according to the invention. Suitable cartridges and methods of filling these is cartridges with the inhalable aerosols containing propellant gas according to the invention are known from the prior art.

### C) Propellant-Free Inhalable Solutions or Suspensions Containing the Combinations of Active Substances According to the Invention

Propellant-free inhalable solutions according to the invention contain for example aqueous or alcoholic, preferably ethanolic solvents, possibly ethanolic solvents in admixture with aqueous solvents. In the case of aqueous/ethanolic solvent mixtures the relative proportion of ethanol to water is not restricted, but the maximum limit is up to 70 percent by volume, more particularly up to 60 percent by volume of ethanol. The remainder of the volume is made up of water. The solutions or suspensions containing 1 and 2, separately or together, are adjusted to a pH of 2 to 7, preferably 2 to 5, using suitable acids. The pH may be adjusted using acids selected from inorganic or organic acids. Examples of particularly suitable inorganic acids include hydrochloric acid, hydrobromic acid, nitric acid, sulfuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid, etc. Preferred inorganic acids are hydrochloric acid and sulfuric acid. It is also possible to use the acids which have already formed an acid addition salt with one of the active substances. Of the organic acids, ascorbic acid, fumaric acid and citric acid are preferred. If desired, mixtures of the above acids may also be used, particularly in the case of acids which have other properties in addition to their acidifying qualities, e.g., as flavorings, antioxidants or complexing agents, such as citric acid or ascorbic acid, for example. According to the invention, it is particularly preferred to use hydrochloric acid to adjust the pH.

According to the invention, the addition of edetic acid (EDTA) or one of the known salts thereof, sodium edetate, as stabilizer or complexing agent is unnecessary in the present formulation. Other embodiments may contain this compound or these compounds. In a preferred embodiment the content based on sodium edetate is less than 100 mg/100 mL, preferably less than 50 mg/100 mL, more preferably less than 20 mg/100 mL. Generally, is inhalable solutions in which the content of sodium edetate is from 0 to 10 mg/100 mL are preferred.

Co-solvents and/or other excipients may be added to the propellant-free inhalable solutions according to the invention. Preferred co-solvents are those which contain hydroxyl groups or other polar groups, e.g., alcohols—particularly isopropyl alcohol, glycols—particularly propyleneglycol, polyethyleneglycol, polypropylene glycol, glycol ether, glycerol, polyoxyethylene alcohols and polyoxyethylene fatty acid esters.

The terms excipients and additives in this context denote any pharmacologically acceptable substance which is not an active substance but which can be formulated with the active substance or substances in the pharmacologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilizers, complexing agents, antioxidants and/or preservatives which guarantee or prolong the shelf life of the finished pharmaceutical formulation, flavorings, vitamins and/or other additives known in the art. The additives also include pharmacologically acceptable salts such as sodium chloride as isotonic agents. The preferred excipients include antioxidants such as ascorbic acid, for example, provided that it has not already been used to adjust the pH, vitamin A, vitamin E, tocopherols and similar vitamins and provitamins occurring in the human body.

Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above are preferably present in concentrations of up to 50 mg/100 mL, more preferably between 5 and 20 mg/100 mL.

Preferred formulations contain, in addition to the solvent water and the combination of active substances 1 and 2, only benzalkonium chloride and sodium edetate. In another preferred embodiment, no sodium edetate is present.

The propellant-free inhalable solutions according to the invention are administered in particular using inhalers of the kind which are capable of nebulizing a small amount of a liquid formulation in the therapeutic dose within a few seconds to produce an aerosol suitable for therapeutic inhalation. Within the scope of the present invention, preferred inhalers are those in which a quantity of less than 100 μL, preferably less than 50 μL, more preferably between 10 and 30 μL of active substance solution can be nebulized in preferably one spray action to form an aerosol with an average particle size of less than 20 μm, preferably less than 10 μm, such that the inhalable part of the aerosol corresponds to the therapeutically effective quantity.

An apparatus of this kind for propellant-free delivery of a metered quantity of a liquid pharmaceutical composition for inhalation is described for example in International Patent Application WO 91/14468 and also in WO 97/12687 (cf. in particular FIGS. 6a and 6b). The nebulizers (devices) described therein are known by the name Respimat®.

The abovementioned examples of the active substances 2 are known in the art. The compounds of formula 1 by contrast are not known in the art.

The examples of synthesis described hereinafter serve to illustrate possible methods of synthesizing the new compounds of formula 1. However, they are intended only as examples of procedures as an illustration of the invention without restricting the invention to the subject-matter described by way of example.

1. A pharmaceutical composition comprising:
(a) a compound of formula 1 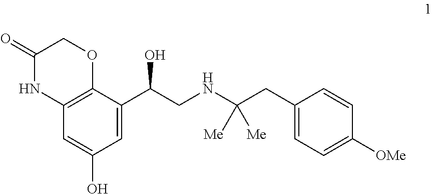 [SMILES: COc1ccc(CC(C)(C)NC[C@H](O)c2cc(O)cc3c2OCC(=O)N3)cc1]
or an enantiomer, mixture of enantiomers, or racemate thereof, or an acid addition salt with pharmacologically acceptable acids thereof; and
(b) another active substance 2, wherein the active substance 2 is a tiotropium salt,
wherein the molar ratio of the compound of formula 1 to the active substance 2 is 1:1 to 5:1.

2. (canceled)

3. (canceled)

4. The pharmaceutical composition according to claim 1, wherein the molar ratio of the compound of formula 1 to the active substance 2 is 1:1 to 3:1.

5. The pharmaceutical composition according to claim 1, wherein the molar ratio of the compound of formula 1 to the active substance 2 is:
a) 1:1 to 1.5:1; or
b) 1.6:1 to 2.0:1; or
c) 2.1:1 to 2.5:1; or
d) 2.6:1 to 3:1.

6. The pharmaceutical composition according to claim 1, wherein the molar ratio of the compound of formula 1 to the active substance 2 is:
a) 1:1; or
b) 2.0:1.

7. (canceled)

8. (canceled)

9. (canceled)

10. The pharmaceutical composition according to claim 1, further comprising a pharmaceutically acceptable carrier.

11. The pharmaceutical composition according to claim 1, wherein the composition does not contain a pharmaceutically acceptable carrier.

12. The pharmaceutical composition according to claim 1, wherein the composition is suitable for inhalation.

13. The pharmaceutical composition according to claim 12, wherein the composition is an inhalable powder, propellant-containing metered-dose aerosol, or propellant-free inhalable solution or suspension.

14. The pharmaceutical composition according to claim 12, wherein the composition is an inhalable powder further comprising a physiologically acceptable excipient selected from monosaccharides, disaccharides, oligo- and polysaccharides, polyalcohols, salts, or mixtures of these excipients.

15. The pharmaceutical composition according to claim 13, wherein the composition is a propellant-driven inhalable aerosol which contains 1 and 2 in dissolved or dispersed form.

16. The pharmaceutical composition according to claim 14, wherein the composition contains as the propellant gas hydrocarbons or halohydrocarbons sue.

17. The pharmaceutical composition according to claim 15, wherein the propellant gas is TG134a (1,1,1,2-tetrafluoroethane) or TG227 (1,1,1,2,3,3,3-heptafluoropropane), or a mixture thereof.

18. The pharmaceutical composition according to claim 13, wherein the composition is a propellant-free inhalable solution or suspension further comprising water, ethanol, or a mixture of water and ethanol.
